(19) United States
(12) Patent Application Publication
(10) Pub. No.: US 2008/0306508 A1
(43) Pub. Date: Dec. 11, 2008

(54) MAGNETIC THERAPEUTIC PAIRED ACCESSORIES

(76) Inventor: Donald Spector, New York, NY (US)

Correspondence Address: DIEHL SERVILLA LLC, 77 BRANT AVE, SUITE 210, CLARK, NJ, 07066, US

(21) Appl. No.: 11/849,405

(22) Filed: Sep. 4, 2007

Related U.S. Application Data
(60) Provisional application No. 60/943,037, filed on Jun. 9, 2007.
(60) Provisional application No. 60/944,109, filed on Jun. 15, 2007.

(51) Int. Cl. A61B 17/00

(52) U.S. Cl. 606/204

(57) ABSTRACT

Provided are methods and articles for magnetic therapy and acupressure which include pieces capable of being connected through a magnetic interaction.

### RELATED U.S. APPLICATION DATA

This application claims the benefit of priority under 35 U.S.C. § 119(e) to U.S. Provisional Application Ser. No. 60/943,037, filed Jun. 9, 2007, and U.S. Provisional Application Ser. No. 60/944,109, filed Jun. 15, 2007, the disclosures of which are hereby incorporated herein by reference thereto.

### TECHNICAL FIELD

This invention relates in general to methods and articles for magnetic therapy and acupressure. Objects capable of forming a magnetic connection are located against a point wherein acupuncture is customarily applied. An article of clothing or a piece of flesh can be located between the objects, so that through the application of force to one article, or the magnetic interaction, pressure is applied to the acupressure point. The invention allows for the discrete application of pressure to the pressure point, thereby providing certain therapeutic benefits.

### BACKGROUND

Acupuncture, a well known practice of Chinese origin, is the puncturing of the body with needles at specific points to relieve pain and affect the curing of certain diseases. The specific points, or acupoints, are well documented in the literature and are known to practitioners in this art. Shiatsu is massage therapy applied with the fingers to those specific areas of the body used in acupuncture. Hence, when pressure is applied to shiatsu meridian lines on the human body, certain desirable benefits are obtained. The meridians are known as the pathway of life energy of the human body.

The present invention utilizes acupoints of oriental medical theory; the same acupoints to which acupuncture is applied. It is well known that there are acupoints located at specific positions of the human body and stimulations of a specific acupoint can result in medical benefits such as relieving pain, improved metabolism and improved health.

Over the past many centuries the benefits of acupressure and massage have been well documented. Massage, as a form of therapy, can be traced back to the ancient Greeks and Romans as an accepted form of medical treatment. One form of massage, acupressure has also been employed by the Chinese for over 5,000 years.

While the end result of acupressure and acupuncture are quite similar, they are different in that acupressure in its truest sense does not utilize needles. In either case it is believed that both systems stimulate the nervous system which releases neurohormones and affects the amounts of neurotransmitters. Moreover, both systems are effective for reducing muscle tension and pain, for at least some period of time. In addition to acupressure and acupuncture, the use of magnetism is also currently in vogue for the treatment of a variety of physical and other conditions.

However, application of acupressure, and particularly acupuncture, was usually done by a professional person at specific locations, such as a doctor's office, health club, or similar location. A person seeking benefit of such therapy usually found difficulties in attempting to treat him or herself.

Accordingly, one or more of the following objects will be achieved by the practice of this invention. It is an object of this invention to provide a method whereby a person seeking the benefit of acupressure, can him or herself apply such pressure to known pressure points on his or her own body with essentially complete discretion and for the most part, without another person being aware of such action. It is a further object of the invention to provide articles whereby a person can discretely apply pressure to predetermined acupoints. Another object is to provide an ornamental article which can be worn as a piece of jewelry, or clothing accessory, which when properly placed and pressed, will apply pressure to the desired acupoint.

### SUMMARY OF THE INVENTION

One or more aspects of the present invention relate to a therapeutic method of providing acupressure to a person by placing one or more pieces on an acupressure point (also called an acupoint). An additional piece is placed against the piece on the acupoint, forming a magnetic connection between the pieces. Application of pressure to one piece, in the direction of the piece located on the acupoint creates pressure on the acupoint. In other aspects of the invention, a piece of clothing is between the pieces.

Another aspect of the invention relates to a therapeutic method of providing acupressure to a person by placing two pieces, which have a magnetic connection between, on an acupoint. The acupoint being between the two pieces so that the magnetic connection of the pieces causes pressure to be applied to the acupoint.

In other aspects of the invention the pieces have at least one side that is substantially planar. In still other aspects one of the pieces is larger than the other piece. In further aspects of the invention an ornamental component is included on one or more the magnetic components. In one or more aspects of the invention a magnet is recessed within the ornamental component. In some aspects, the magnet is recessed so that it is practically flush with the surface of the magnetic piece.

In other aspects of the invention, the therapeutic method includes a magnetic piece or pieces with a soft material on the surface which contacts the skin. Additional aspects of the invention have a protective layer coating at least one of the pieces. In still further aspects, a transdermal patch is applied to a piece which is in contact with the skin, thereby allowing, among other things, application of a medicine, or herbal, to the skin.

Additional aspects of the invention relate to a kit for magnetic therapy and acupressure comprising at least two pieces which are connected through a magnetic interaction. In one or more aspects of the invention, at least one piece further comprises a soft material, a means of delivering medication, an ornamental component and/or a magnet which is flush with the surface of a piece.

In further aspects of the invention, the pieces have at least one surface that is substantially planar. In additional aspects, the area of one of the substantially planar surfaces is larger than the substantially planar surface on the other piece.

### BRIEF DESCRIPTION OF THE DRAWINGS

FIG. 1 shows an article having two magnetic components with one being larger than the other.

FIG. 2 shows a larger magnetic component with a magnet located within a recess.

FIG. 3 shows an article having two magnetic components, where the smaller component has a soft pad and the larger component has an ornamental decoration.

### DETAILED DESCRIPTION OF THE INVENTION

References to an “object”, “piece”, “magnetic object” or “magnetic piece” refer to a physical component which either contains a magnet, is magnetic, or is capable of interacting with a magnet to form a magnetic connection.

One or more aspects of the present invention relate to a therapeutic method of providing acupressure and/or magnetic therapy to a person by placing an object, or piece, on an acupressure point (also called an acupoint). An additional object is placed against the piece on the acupoint, forming a magnetic connection between the pieces.

In other aspects of the invention, force is applied to the piece not in contact with the skin, in the direction of the piece in contact with the skin. The contact of this, and other aspects of the invention, causes pressure to be applied to the skin through the connection of the magnetic pieces.

In further aspects of the invention, a piece of clothing is located between the pieces. In these aspects, the piece that is in contact with the acupoint may be, but is not required to be, hidden from view from the casual observer, allowing someone to discretely employ acupressure treatment and magnetic treatment.

In other aspects of the invention, the pieces are placed in such a manner so that there is a portion of skin located between them. The magnetic interaction between the pieces can cause the application of pressure to the point between. As a non-limiting example, the pieces can be placed on an acupoint of an earlobe where the magnetic connection will pull the piece toward each other, thereby squeezing, and applying, pressure the acupoint.

In still further aspects of the invention, one piece is larger than the other. Pressure is defined as the force per unit area applied to a surface in a direction perpendicular to that surface. If an equal force is applied over two different areas, two different pressures will result. This fundamental relationship can be exploited, as in some aspects of this invention, to apply a relatively large pressure with only a minimal amount of force. For example, if a small surface area object is placed on the skin with a larger surface area object placed on the small surface area object, the force applied to the larger surface area object will be transferred to the smaller surface area object, thereby causing pressure on the skin which is greater than that which would be felt if the larger surface area object only was used.

In additional aspects, at least one piece has a soft material on the surface. The soft material on the surface can be used to make the piece more comfortable against the skin, or to help prevent necrosis of the tissue beneath the piece.

In further aspects of the invention, an ornamental component can be applied to at least one of the pieces. This can be used to decorate the piece, making it appear to be, for example, a piece of jewelry, a brooch or an earring. The ornamental piece of this and other aspects can be made of a metal capable of interacting with a magnet or can contain a magnet. In other aspects, the ornamental piece contains a magnet which is flush with the surface of the ornamental piece. Further aspects of the invention have the magnet fully contained within the ornamental piece.

In other aspects, the pieces contain at least one surface that is substantially planar. The area of the substantially planar surface on one piece is larger than the substantially planar surface on the other piece. Preferably, the larger area surface is greater than about 1.5 times the area of the smaller surface. Even more preferably, the larger surface area is greater than about 3 times the area of the smaller surface.

Further aspects of the invention have at least one piece encased within a protective layer. Magnets, depending on the material of construction, can be susceptible to rust and breakage due to being brittle, amongst other limitations. The protective layer can be, but is not limited to, a vinyl coating or other material which would prevent rust from forming or breakage of the magnet. Furthermore, the protective layer may serve to help prevent necrosis of the underlying tissue by providing a broader surface area.

In additional aspects of the invention, one or more pieces have a surface which can apply medication to the skin. This can be accomplished through, for example, a transdermal patch. Medications or botanicals can be applied directly to the acupoint. The pressure applied to the acupoint will help increase the absorption of the medication or botanical. The medication delivery portion of various aspects of the invention can be replaced to allow for reuse of the pieces. In a case where aspects of the invention are applied to the ear lobes, the constant pressure of the magnets and the transdermal patch apply pressure to the point, apply magnetic therapy and herbal or medical treatments at the same time. Obviously, on any part of the body the application of pressure will increase the effectiveness of the acupressure, the magnetic therapy and the transdermal transmission.

Other aspects of the invention pertain to a kit for providing magnetic therapy and acupressure comprising two pieces which are connected through a magnetic interaction. In other aspects of the invention, the kit includes at least one piece containing one or more of the following: a soft material, an ornamental component, a magnet recessed nearly flush within the ornamental piece, a protective layer, a means for delivering medication and/or one piece being larger than the other.

In other aspects, the pieces contain at least one surface that is substantially planar, with the area of the substantially planar surface of one piece being larger than the area of the substantially planar surface of the other piece. Preferably, the area of the larger surface is at least about 1.5 times that of the smaller piece. Even more preferably, the area of the larger surface is at least about 3 times that of the smaller piece.

In further aspects of the invention, the kit includes pieces which are adapted for placement of an article of clothing between the pieces. In still further aspects, the kit includes pieces which are adapted for placement of the pieces around a piece of flesh.

In other aspects of the invention, the kit comprises two pieces where one piece has a soft material on one or more sides. Further aspects have one piece larger than the other piece. In additional aspects, the pieces include one or more of an ornamental component, a magnet recessed flush with the ornamental component, a protective layer, a means for delivering medication, and a substantially planar surface.

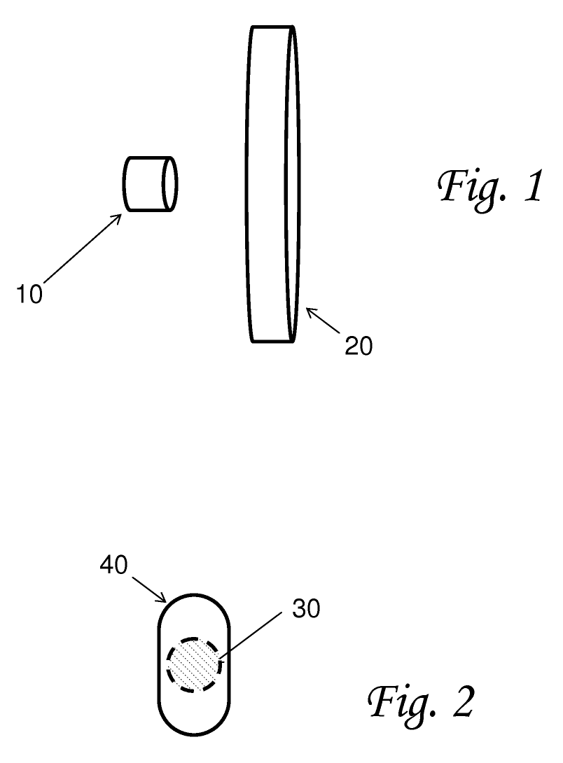

FIG. 1 shows a small piece 10 and a large piece 20, both of which are capable of a magnetic interaction. At least one of the pieces is a magnet, or contains a magnet.

FIG. 2 shows a magnet 30 enclosed within a protective layer 40. The protective layer can be, but is not limited to, a vinyl coating. The dotted line of the magnet 30 indicates that the magnet 30 is not visible because it is completely encased within the protective layer.

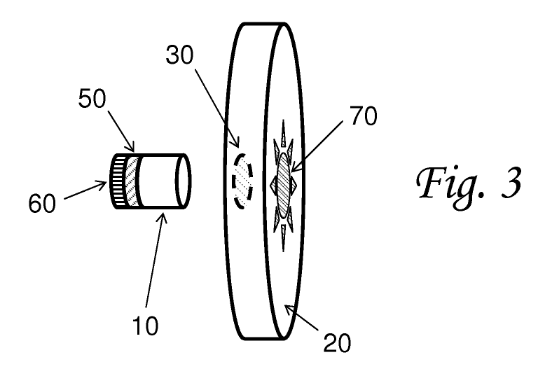

FIG. 3 shows a small piece 10 and a large piece 20, both of which are capable of a magnetic interaction. At least one of the pieces is a magnet, or contains a magnet 30. A magnet 30 is shown, but is not necessarily present, recessed substantially flush with the surface of the large piece 20, on the side facing the small piece 10. The dotted line of the magnet 30 indicates that the magnet 30 is not visible from the orientation of the figure. A pad 50 is located on a side of the small piece. The pad 50 can be, for example, a soft coated pad or a pad for connecting a medication delivery device 60. The medication delivery device 60 can be attached to either the small piece 10 or the large piece 20 with or without the pad 50. An ornamental component 70 is shown on the front of the large piece 20, but can be located on either piece. The ornamental component 70 shown is an image, but can be any type of ornamentation, such as gemstones and figures.

Although the invention herein has been described with reference to particular embodiments and aspects, it is to be understood that these embodiments and aspects are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A therapeutic method of providing magnetic therapy and acupressure to a person having a skin comprising:
placing a first piece on an acupoint; and
placing a second piece near the first piece so that there is a magnetic connection between the first piece and the second piece;
wherein at least one piece comprises a magnet.

2. The therapeutic method of claim 1 wherein pressure is applied to the acupoint by application of force to the second piece in the direction of the first piece.

3. The therapeutic method of claim 1 wherein pressure is applied to the skin by the magnetic interaction between the first and second pieces.

4. The therapeutic method of claim 1 wherein one piece is larger than the other piece.

5. The therapeutic method of claim 1 wherein at least one piece has a surface with a soft material.

6. The therapeutic method of claim 1 wherein at least one piece further comprises an ornamental component.

7. The therapeutic method of claim 6 wherein a magnet is recessed substantially flush within the surface of the ornamental component.

8. The therapeutic method of claim 1 wherein an article of clothing is located between the first piece and the second piece.

9. The therapeutic method of claim 1 wherein skin is located between the first piece and second piece.

10. The therapeutic method of claim 1 wherein the first piece and the second piece have at least one surface that is substantially planar and one piece has an area greater than about 1.5 times the area of the other piece.

11. The therapeutic method of claim 1 wherein at least one piece is encased within a protective layer.

12. The therapeutic method of claim 1 wherein at least one piece further comprises a means for delivering medication.

13. A kit for providing magnetic therapy and acupressure comprising:
a first piece; and
a second piece;
wherein the first piece and the second piece are connected through a magnetic interaction.

14. The kit of claim 13 wherein a surface of at least one piece has a soft material.

15. The kit of claim 13 wherein at least one piece further comprises an ornamental component.

16. The kit of claim 15 wherein a magnet is recessed substantially flush with a surface of the ornamental component.

17. The kit of claim 13 wherein the first piece and the second piece have at least one surface that is substantially planar and one piece has an area greater than about 1.5 times the area of the other piece.

18. The kit of claim 13 wherein at least one piece is encased within a protective layer.

19. The kit of claim 13 wherein at least one piece further comprises a means for delivering medication.

20. A kit for providing magnetic therapy and acupressure comprising:
a first piece; and
a second piece;
wherein the first piece has a soft material on one side.

21. The kit of claim 20 wherein one piece is larger than the other piece.

22. The kit of claim 20 wherein at least one piece has an ornamental component and a magnet.

23. The kit of claim 20 wherein at least one piece is encased within a protective layer.

24. The kit of claim 20 wherein at least one piece further includes a means for delivering medication.

25. The kit of claim 20 wherein the first piece and the second piece have a surface that is substantially planar and one piece has an area greater than about 1.5 times the area of the other piece.
